(19) United States
(12) Patent Application Publication
(10) Pub. No.: US 2011/0060296 A1
(43) Pub. Date: Mar. 10, 2011

(54) Wound covering and method for treating a wound

(76) Inventors: Judson Threlkeld, Camas, WA (US); Joseph Hare, Chelan, WA (US)

(21) Appl. No.: 12/584,753

(22) Filed: Sep. 10, 2009

(51) Int. Cl. A61L 15/44; A61F 13/02; A61F 13/15; A61M 35/00

(52) U.S. Cl. 604/290; 602/42; 604/358; 602/48; 604/305

(57) ABSTRACT

A wound covering is described herein and which includes a flexible substrate having first and second portions which are affixed together, and moveable one relative to the other, and wherein the second portion is, at least in part, optically transparent, and permits the viewing of a wound site on a patient; and an at least partially rigid member mounted on the first portion, and extends outwardly relative to the first portion.

### TECHNICAL FIELD

The present invention relates to a wound covering, and method for treating a wound, and more specifically to a wound covering which includes a pressure pad region; and an absorbent wound covering portion which can be observed by a care giver when the wound covering portion is being utilized on a patient.

### BACKGROUND OF THE INVENTION

The prior art is replete with numerous examples of various wound coverings, including hemostats of various designs, and which have been utilized through the years to aid clinicians in the treatment of various maladies. For example, with respect to various wound dressings, various designs have been marketed which allow a clinician, or other health care provider to monitor the site of a wound following a surgical intervention. For example, the Office's attention is directed to the hemostatic bandage marketed by a company identified as Vascular Solutions under the trademark D-STAT DRY CLEAR and which provides a clear bandage which allows for the clear visualization of a wound site. This product has reinforced borders which reportedly provide for more precise placement of the bandage in comparison to other clear bandages. Further, other pressure assisted dressings are marketed currently for various clinical applications. For example, the Office's attention is directed to the pressure assisted dressing marketed under the trademark SAFEGUARD and which is provided by a company named DataScope. The SAFEGUARD pressure assisted dressing provides an adhesive portion which is attached to the skin of a patient, and an inflatable balloon which can apply an adjustable amount of pressure in order to assist in the closure of a wound site. Still further, the SAFEGUARD product allows a clinician to continue to visualize the wound site while pressure is being applied.

In addition to the foregoing products that are currently available for purchase, it has long been known to provide various hemostat arrangements which provide a means by which a flowable substance may be provided to the hemostat while it is being utilized. In this regard, the Office's attention is directed again to the commercial product provided by the company Vascular Solutions, and which is identified by the trademark, “D-STAT Flowable Hemostat”.

While the prior art devices and methodologies used heretofore have been widely embraced and are generally successful for their intended purposes, clinicians have continued to search for a convenient wound covering which provides a means by which pressure can be selectively applied, and thereafter the wound site can be monitored while also providing a convenient means whereby selected medicaments may be provided to the wound site without removing the wound covering.

A wound covering and method for treating a wound is the subject matter of the present application.

### SUMMARY OF THE INVENTION

A first aspect of the present invention relates to a wound covering which includes a flexible substrate having first and second portions which are affixed together, and moveable one relative to the other, and wherein the second portion is at least, in part, optically transparent, and permits the viewing of a wound site on a patient; and an at least partially rigid member mounted on the first portion, and extending outwardly relative to the first portion.

Another aspect of the present invention relates to a wound covering, which includes a flexible substrate having an inwardly facing surface which is coated, at least in part, by an adhesive, and an outwardly facing surface, and wherein the flexible substrate has a first portion, and a second portion, and wherein the second portion is, at least in part, optically transparent, and is operable to cover a site of a wound on a patient's body; a pressure pad mounted on the inwardly facing surface of the first portion of the flexible substrate, and which is oriented in rested relation on a patient's body, and wherein a clinician applies force to the pressure pad to achieve a desired medical outcome; and an absorbent wound covering portion which is oriented in covering relation relative to the site of the wound on the patient's body, and which is secured in place by the second portion of the flexible substrate.

Still further, the present invention relates to a method for treating a wound, which includes the steps of providing a flexible substrate having opposite inwardly and outwardly facing surfaces and which defines a first portion, and a second portion which are moveably coupled together; applying an adhesive to at least a portion of the inside facing surface of the flexible substrate; mounting a pressure pad on the inside facing surface of the first portion of the flexible substrate; positioning the pressure pad in an anatomically correct position on a patient's body and adhesively affixing the first portion of the flexible substrate to the patients body; after the step of positioning the pressure pad, orienting the second portion of flexible substrate in spaced relation relative to the patient's body; after the step of orienting the second portion of the flexible substrate in spaced relation relative to the patient's body, implementing a medical procedure which creates a wound site in the patient's body which is located in spaced relation relative to the pressure pad; moving the second portion of the flexible substrate into covering relation relative to the wound site, and adhesively affixing it to the patient's body; and positioning an absorbent wound covering portion over the wound site, and in sandwiched relation therebetween the patient's body, and the inside facing surface of the second portion of the flexible substrate prior to adhesively affixing the second portion of the flexible substrate to the patient's body.

These and other aspects of the present invention will be described in greater detail hereinafter.

### BRIEF DESCRIPTION OF THE DRAWINGS

Preferred embodiments of the invention are described below with reference to the following accompanying drawings.

FIG. 1 is a partial, top plan view of a first form of the present invention.

FIG. 2 is a transverse, vertical, sectional view taken along line 2-2 of FIG. 1.

FIG. 3 is a partial, top plan view of a second form of the present invention with some underlying structures shown in phantom lines.

FIG. 4 is a transverse, vertical, sectional view taken along line 4-4 of FIG. 3.

FIG. 5 is a side elevation view of the present invention being employed in a medical procedure.

FIG. 6 is a partial, top plan view of a third possible form of the present invention.

FIG. 7 is a top plan view of the first form of the present invention with some underlying structures shown in phantom lines.

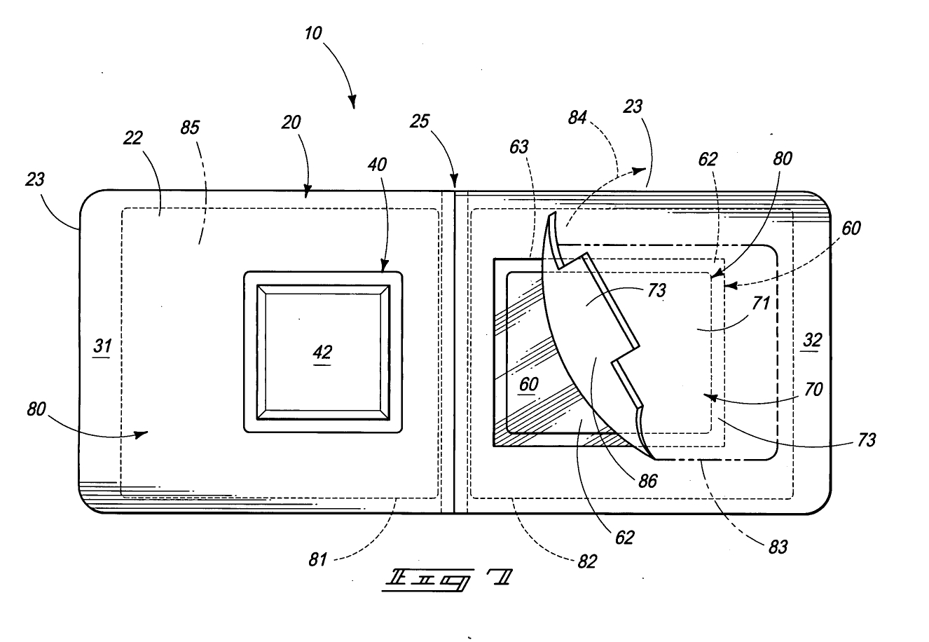

FIG. 8 is a bottom plan view of the first form of the present invention with some underlying structures shown in phantom lines.

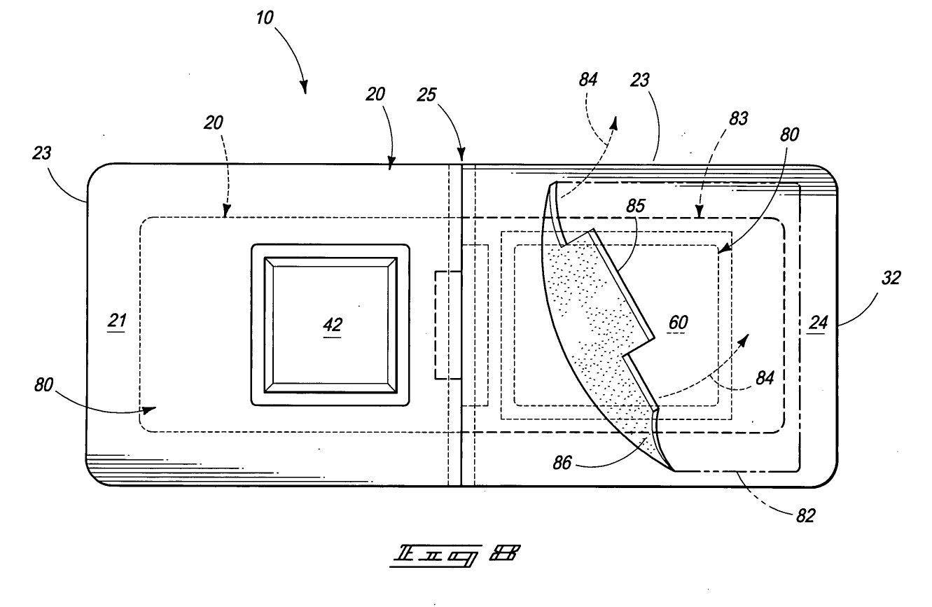

FIG. 9 is a top plan view of another form of the present invention.

FIG. 10 is a transverse, vertical, sectional view taken along line 10-10 of FIG. 9.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

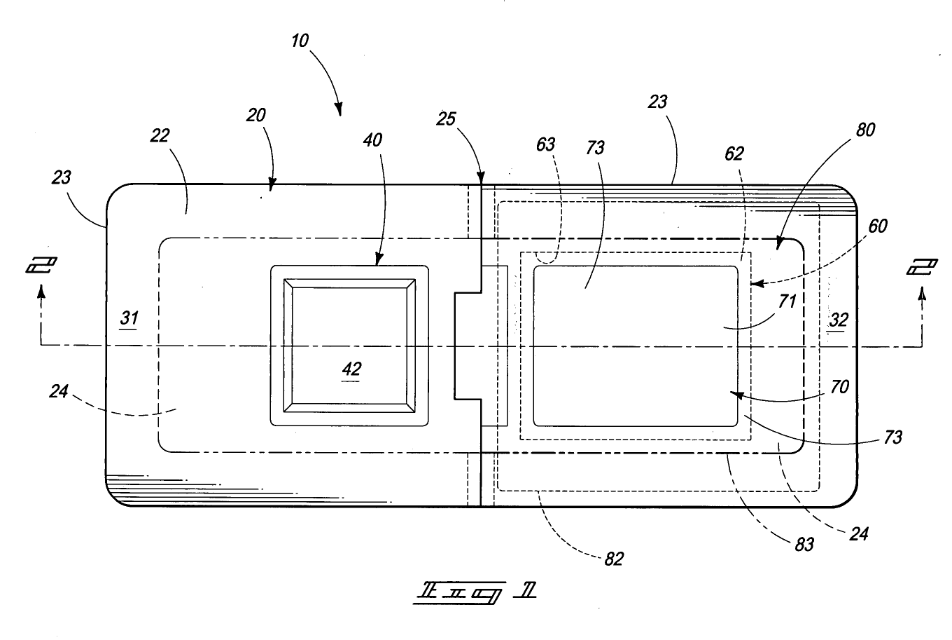

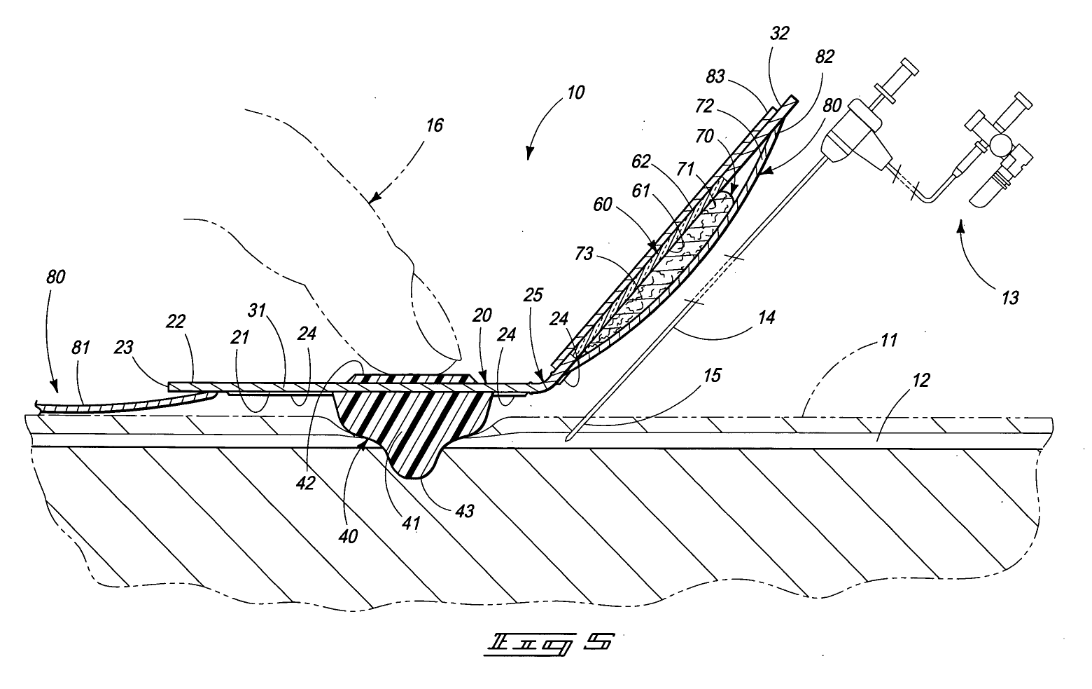

The present invention as noted above relates to a wound covering and a method for treating a wound. The present invention is generally indicated by the numeral 10 in FIG. 1, and following. As should be understood, the wound covering is operable to be placed on the skin surface of a patient's body 11. This is seen most clearly by reference to FIG. 5. As seen in that drawing, it will be understood that in various medical procedures, a clinician will need to gain access to a blood vessel or other body cavity, or region 12 located below the surface of the patient's body 11. As will be understood from that view, often the clinician will employ a medical instrument which is generally indicated by the numeral 13, and which has a needle 14 extending therefrom and which is utilized to puncture the skin surface of the patient's body 11 to gain access to, for example, a blood vessel 12 positioned therebelow to remove body fluids, or to inject medicaments or other treatment substances. As should be understood, the location where the needle 14 crosses the skin surface of the patient's body 11 creates a wound site 15 which needs to be treated once the medical instrument 13 is removed to prevent bleeding, bacterial infection and to further promote healing. It has long been known that providing pressure at a given distance which is spaced from the wound site 15 often facilitates certain medical procedures, and facilitates the proper formation of the wound site 15 for the procedure as shown in FIG. 5. As seen in FIG. 5, this pressure is being applied by a clinician's finger which is generally indicated by the numeral 16.

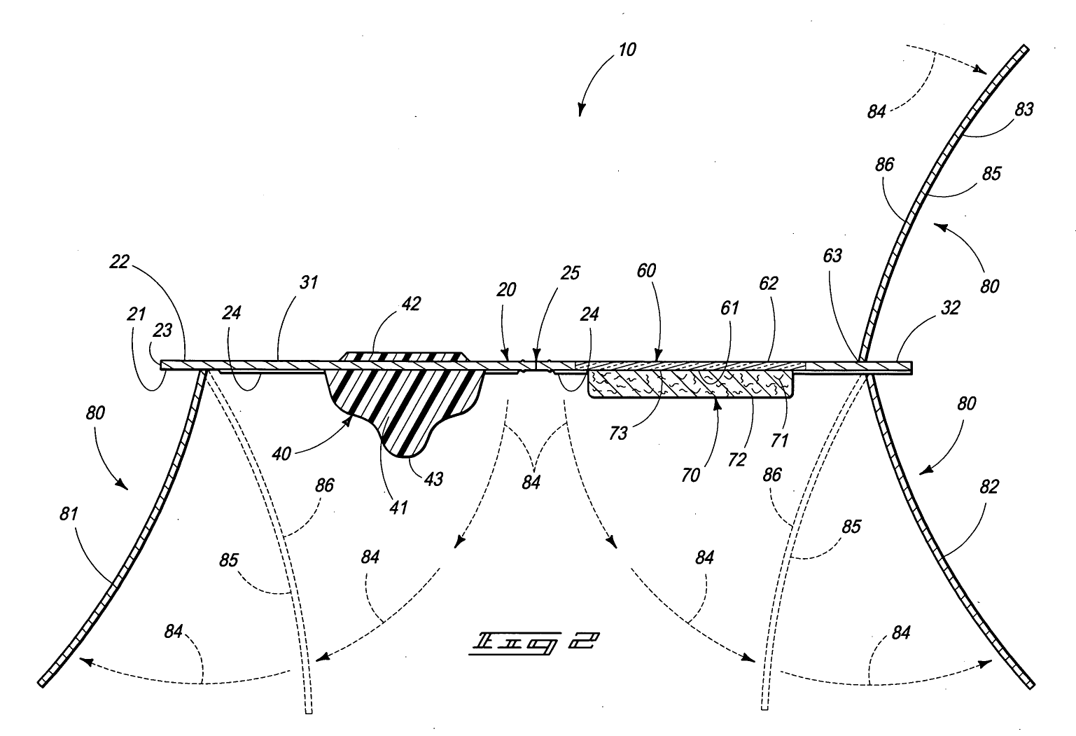

Referring now to FIG. 1, which shows one of form of the invention, the wound covering 10 is fabricated from a flexible substrate which is indicated by the numeral 20. The flexible substrate 20 has a first, inwardly facing surface 21 (FIG. 2), and an a second, outwardly facing surface generally indicated by the numeral 22. The flexible substrate is defined by a peripheral edge 23. Further, the inwardly facing surface 21 is covered, at least in part, by an adhesive layer 24 which is effective in securing the flexible substrate to the body surface 11 of the patient's body as indicated in FIG. 5. In addition, the flexible substrate 20 is defined by a first portion 31, and a second portion 32, which are moveably affixed together at a crease, joint or hinge 25, and are further moveable, one relative to the other, as seen in FIG. 5. Still further, as will be appreciated by a study of FIG. 1, and following, the second portion 32 has an optically transparent portion as will be described below. This permits the viewing of a wound site 15 on a patient when the wound covering is employed following a medical procedure which creates a wound site 15.

In the form of the invention as seen in FIG. 2, the wound covering 10 includes an at least partially rigid member, or pressure pad 40 which is mounted on the first portion 31, and which extends outwardly relative to the first inwardly facing surface 21. As seen in the cross-sectional view of FIG. 2, the rigid member or pressure pad 40 has a main body 41 which includes an uppermost portion 42 which may, in this form of the invention, extend slightly above the second outwardly facing surface 22 so as to provide a convenient tactile orientation point for a clinician's hand or finger 16 (FIG. 5). Further, the pressure pad 40 has a protruding, lowermost portion 43 which is positioned in contact with the surface of the patient's body 11 as seen in FIG. 5. As discussed above, and as seen in FIG. 5, a clinician's finger 16 may press upon the uppermost portion 42 in order to transmit force through the rigid member or pressure pad 40 to the underlying skin surface of the patient's body 11 for various medical purposes including to aid in the formation of an appropriate wound site 15, by the medical instrument 13, as illustrated in that view.

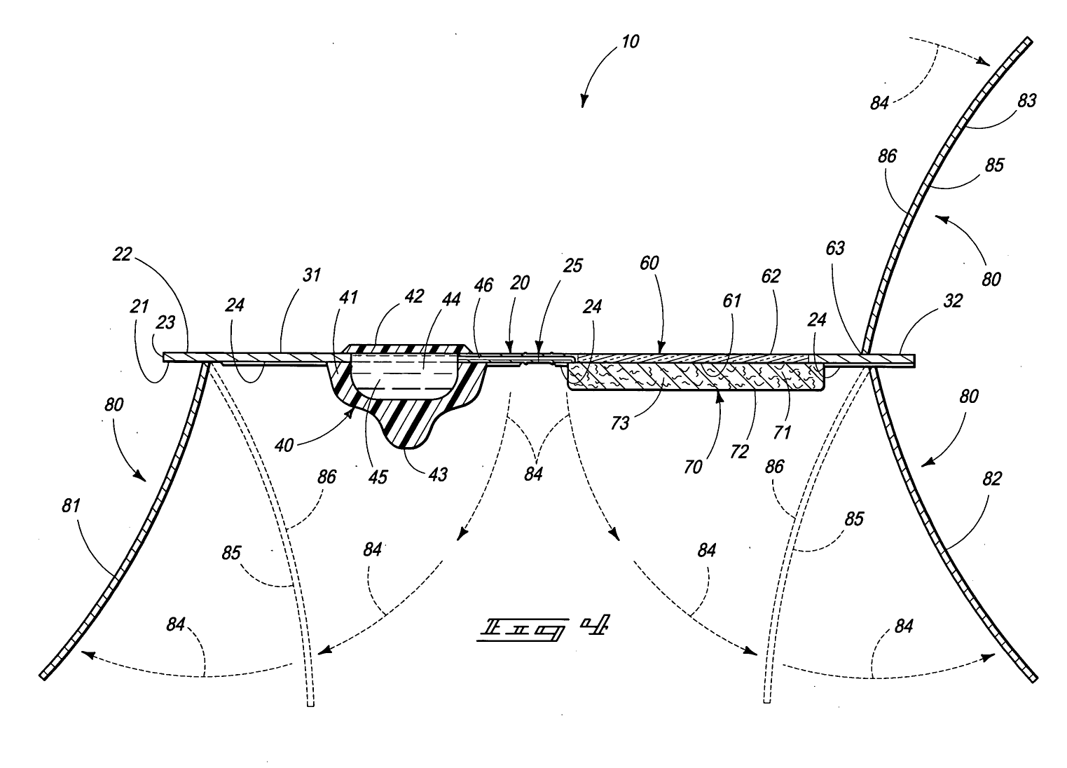

Referring now to FIG. 4, it will be seen that in a second possible form of the invention 10, as illustrated, the rigid member or pressure pad 40 may have an internal cavity 44 formed therein. The internal cavity 44 defines a storage space to receive a flowable medicament 45 which might include, for example, a bactericidal solution, or pain control substance, or other suitable clinical solution which may be useful in the treatment of the wound site 15. As should be understood, a fluid passageway or conduit 46 is formed in the pressure pad 40, and the flexible substrate 20, and couples the internal cavity 45, with the second portion 32 of the flexible substrate 20, and more specifically the first inwardly facing surface 21 thereof. As best imagined by reference to FIG. 4, it will be understood that a given amount of pressure applied by a clinician's finger 16, for example, on the pressure pad 40 is effective to force or cause the medicament 45 to flow through the fluid passageway 46, and specifically, the upper portion thereof 42, so as to deform same and cause the flowable medicament 45 to be forced out of the cavity 44, and be delivered to the skin surface of the patient's body 11 which underlies, or is covered by, the second portion 32 of the flexible substrate 20. Therefore, in at least one form of the invention, the rigid member or pressure pad 40 operates to firstly, transmit force from a clinician's finger to the underlying surface of the patient's body 11 to achieve some desired medical benefit; and secondly, is effective to cause a medicament or other flowable substance that has been delivered into the internal cavity 44, to thereafter be squeezed or otherwise forced out of same and be delivered to the wound site 15 which is underlying the second portion 32 of the flexible substrate 20. When a clinician is employing the wound covering as seen in FIG. 5, it will be appreciated that the bending or folding of the second portion 32, relative to the first portion 31 would have the effect of temporarily blocking the passageway 96 which formed in the flexible substrate 20, and which extends to the second portion 32 of the substrate 20.

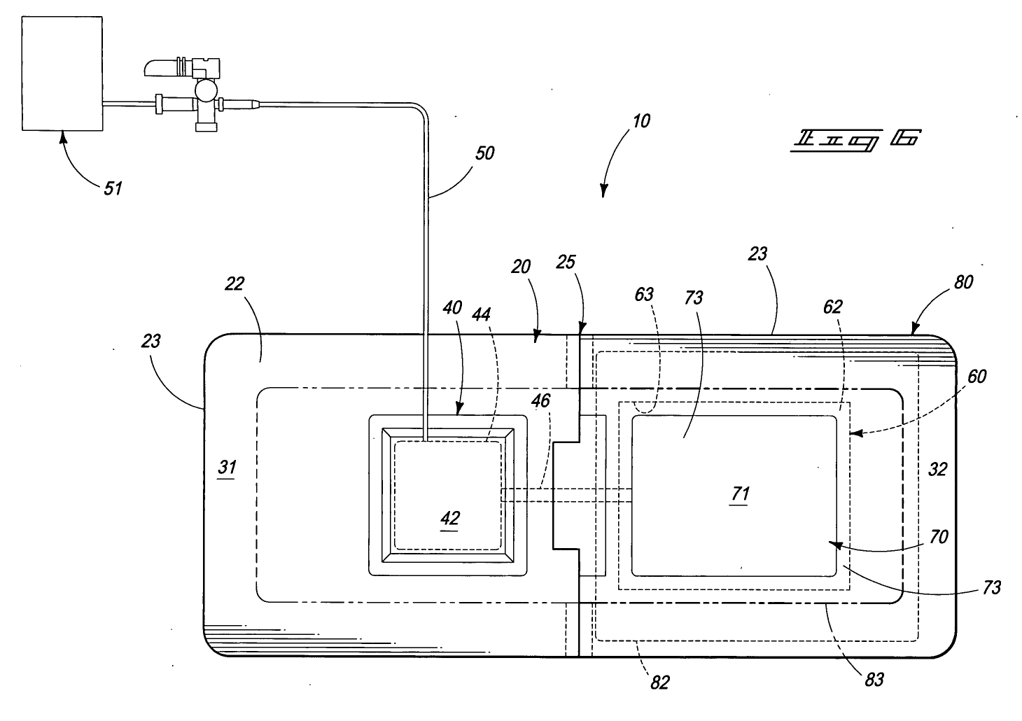

In another possible form of the invention as seen in FIG. 6, a conduit 50 is coupled in fluid flowing relation relative to the internal cavity 44 of the rigid member or pressure pad 40. The conduit 50 is operable to be coupled to a source of the medicament 51 to be delivered to the internal cavity 44. This arrangement permits the medicament or other flowable substance 45 to be changed; supplemented or resupplied based upon the condition of the patient or changing wound conditions. Moreover, this arrangement provides a convenient means for delivering a source of a medicament or plurality of medicaments or other flowable treatment substance to a wound site 15 without removing the second portion 32 from its covering relationship relative to the skin surface of the patient's body 11. This feature substantially prevents the bacterial contamination or other disturbance of the wound site 15.

As seen in FIGS. 1, 2, and following, it will be understood that the wound covering 10 includes an optically transparent window or region 60 which permits the viewing of the wound site 15 on a patient. In this regard, the transparent window 60 is made integral with the second portion 32, and has an inside facing surface 61, and an outside facing surface 62. The inside facing surface 61 may, in one form of the invention, have an adhesive coating applied thereto. The transparent window 60 is defined by a peripheral edge 63. As best seen by reference to FIG. 2 and following, it will be understood that an absorbent wound covering portion 70 is provided and positioned in juxtaposed relation relative to the transparent window 60 and is held in a sandwiched relationship therebetween the surface of the patient's body 11, and the first inwardly facing surface 21 of the second portion 32. The absorbent would covering portion has a main body 71, with an inwardly facing surface 72, and an outwardly facing surface 73. Because the first inwardly facing surface 21 has an adhesive coating 24 applied thereto, the absorbent wound covering portion 70 is secured to the second portion 32 and is further held in place over the top of the wound site 15. In one possible form of the invention 10, the absorbent wound covering portion 70 may be first supplied or coated with a medicament or other solution for treating the wound site 15, and then may be placed into covering relation relative to the wound site 15 prior to moving the second portion 32 into covering relation relative to the wound site 15. In another possible form of the invention, the absorbent wound covering 70 may be adhesively affixed by its outside facing surface 73 to the inside facing surface 61 of the transparent window 60 (FIG. 5). Still further, the main body 71 of the absorbent wound covering portion may be supplied with a medicament, such as a bactericidal solution, pain reliever, or other kind of flowable treatment solution prior to being brought into contact with the wound site. Additionally, the flowable treatment solution may be provided from the reservoir 44 as seen in FIG. 4 and which is defined by the structure 40. Any solution carried by the main body of the absorbent wound covering portion 71 would be dispensed by way of its inside facing surface 72 directly to the wound site 15 to achieve the medical benefits desired.

Referring now to FIG. 2 and following, it will be seen that the wound covering 10, and more specifically the flexible substrate thereof 20 has a multitude of removable cover portions 80 mounted thereon, and which maintains portions of the inwardly or outwardly facing surfaces 21 and 22 in a substantially sterile condition prior to use. Further, it will be understood by a study of FIG. 2, and following, that the removable cover portions 80 are selectively removed prior to placing the flexible substrate 30 in resting relation on the body surface 11 of the patient as seen in FIG. 5. As seen in the drawings, the removable cover portions 80 include individual covers here labeled 81, 82 and 83, respectively, and which can be independently, and separately removed while the remaining portions of the removable cover 80 remain in covering relation relative to the inwardly and/or outwardly facing surfaces 21 and 22 of the flexible substrate 20. As best understood by a study of FIG. 5, the portion of the removable cover, here indicated by the numeral 81, is removed prior to placing the first portion 31 of the flexible substrate in resting relation on the body surface 11 of the patient, and more specifically at a location which is spaced from the wound site 15. The respective removable covers 80, are typically moveable along an arcuately shaped path of travel 84 out of contact with the underlying surface of the flexible substrate 20 that it protects. Each removable cover 80 has an outside facing surface 85, and an opposite inside facing surface 86 which is coated so as to easily release from the underlying layer of adhesive 24 which is applied to the flexible substrate 20 as earlier discussed. Each cover may be easily detached at a weakened connection point from the flexible substrate 20, and then discarded.

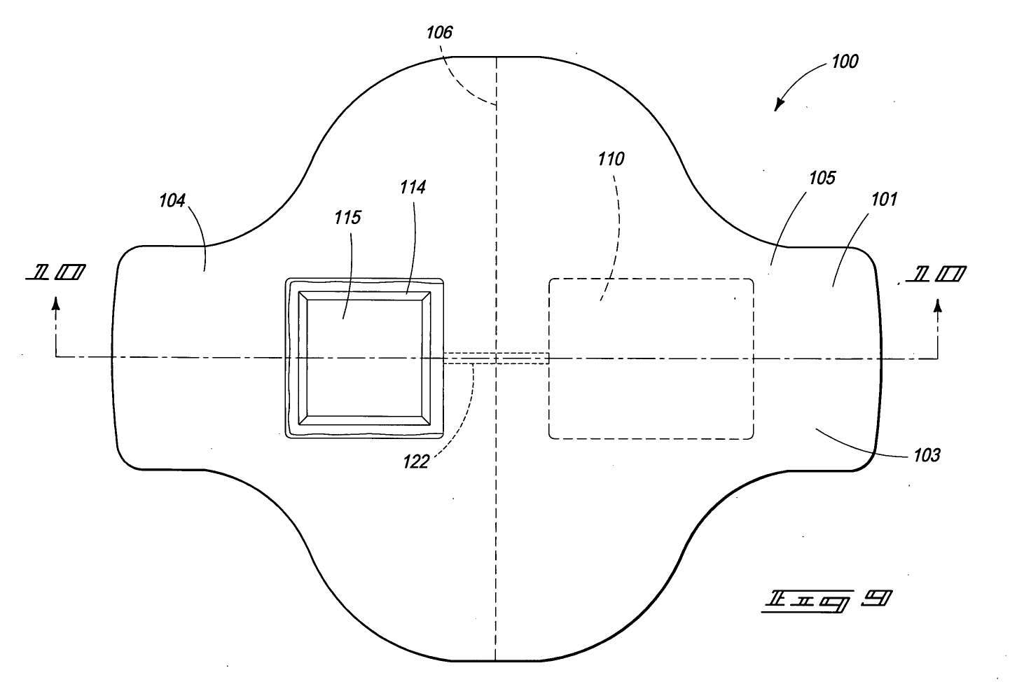

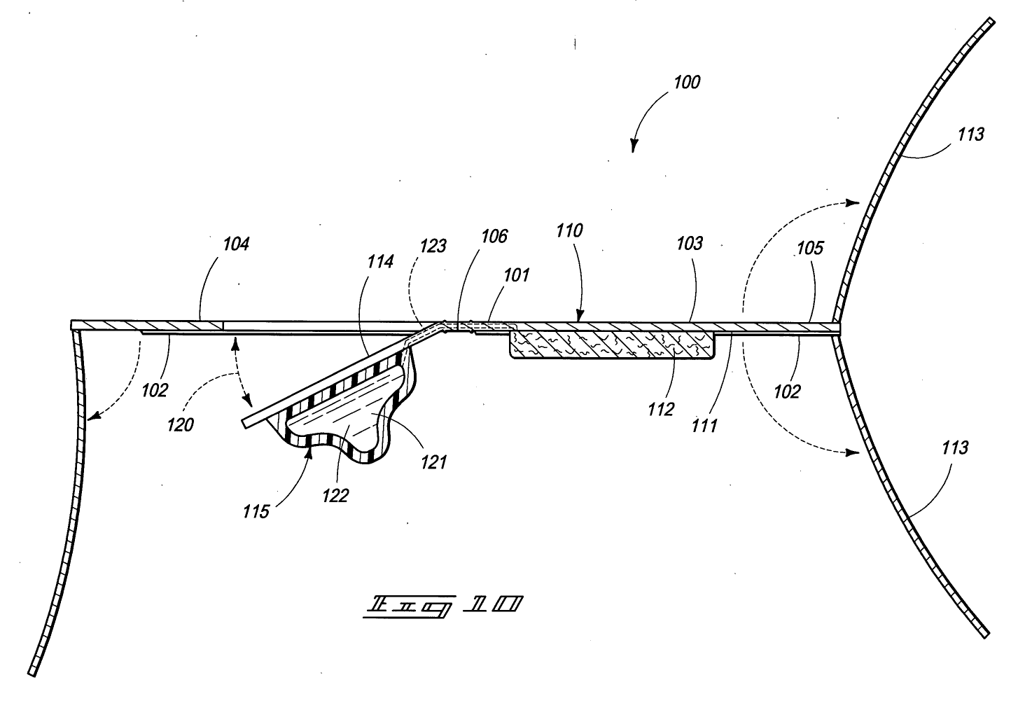

Referring now to FIGS. 9 and 10, another possible form of the invention is shown, and designated by the numeral 100. In this form of the invention, a flexible substrate 101 is provided which again has inwardly and outwardly facing surfaces 102 and 103, respectively. Again the flexible substrate has a first portion 104, and a second portion 105 which are joined together at a flexible joint or hinge 106. Similar to the earlier forms of the invention, an optically transparent window 110 is provided which allows a clinician to visually observe an underlying wound site 15. Further, a coating of adhesive 111 is applied to the inwardly facing surface 102. As with the other forms of the invention, a wound covering 112 is provided and placed over a wound site 15 and is located so as to be seen through the optically transparent window 110. Further, and similar to the other forms of the invention, a plurality of releasable covers 113 are provided, and which can be removed from the flexible substrate 101 so as to expose the underlying surface(s). In this form of the invention, however, the first portion 104 has a moveable tab 114 which has affixed thereto a rigid member or pressure pad 115. As seen in FIG. 10 the tab may be moved downwardly along an arcuately shaped path of travel 120. Again, as in one of the earlier forms of the invention, the pressure pad 115 may be formed so as to have a cavity 121 and which contains a given flowable treatment solution 122 which is to be applied, or otherwise supplied to the wound covering 112 by means of the passageway or conduit 123 which is formed in, or mounted on the flexible substrate 101. This form of the invention provides some advantages over the other forms of the invention because pressure may be supplied by a clinicians finger, for example, to the tab 114, and to the patients body, but the same pressure will not tend to cause the adjacent first portion 104 of the flexible substrate to pull needlessly on the adjacent skin areas of the patient as the pressure is being applied. This will provide increased comfort to the patient as the wound site 15 is being created.

### Operation

The operation of the described embodiments of the present invention are believed to be readily apparent and are briefly summarized at this point.

The several forms of the present invention in its broadest aspect relates to a wound covering 10 which includes a flexible substrate 20 having first and second portions 31 and 32, and which are affixed together, and which are further moveable one relative to the other, and wherein the second portion 32 is, at least in part, optically transparent 60, and permits the viewing of a wound site 15 on a patient 11; and an at least partially rigid member 40 is mounted on the first portion 31, and extends outwardly relative to the first portion 31, and which is operable to engage a patient for various medical purposes as seen most clearly by reference to FIG. 5. Still further, the partially rigid member 40 may be employed to carry a source of a flowable substance which may be dispensed to the wound site 15.

Moreover, in its broadest aspect the wound covering 10, and more specifically the flexible substrate 20 thereof has an outwardly facing surface, and an inwardly facing surface 22 and 21, respectively, and which rest on a body surface of a patient 11, and further, the at least partially rigid member 40 is mounted on the flexible substrate 20, and extends outwardly relative to the inwardly facing surface 21 of the first portion 31. Still further, the inwardly facing surface 21 is covered, at least in part, by an adhesive 24 which is effective in securing the flexible substrate 20 to the body surface of the patient 11. In addition to the foregoing, the wound covering 10 includes a multiplicity of removable covers 80 which are mounted thereon, and which maintain the inwardly and/or outwardly facing surfaces 21 and 22 in a substantially sterile condition. As earlier discussed, the removable covers 80 are individually removed prior to placing the flexible substrate 20 in resting relation on the body surface 11 of the patient. In one form of the invention, the respective removable covers 80 include a plurality of portions 81-83 which can be individually separately removed while the remaining portions of the removable cover 80 remain in covering relation relative to the inwardly and/or outwardly facing surfaces 21 and 22 of the flexible substrate 20. In the form of the invention as illustrated in FIGS. 1 and 2, the at least partially rigid member 40 is a pressure pad which rests on a body surface of a patient 11, and wherein a clinician 16 applies force to the pressure pad 40 to achieve a desired medical outcome.

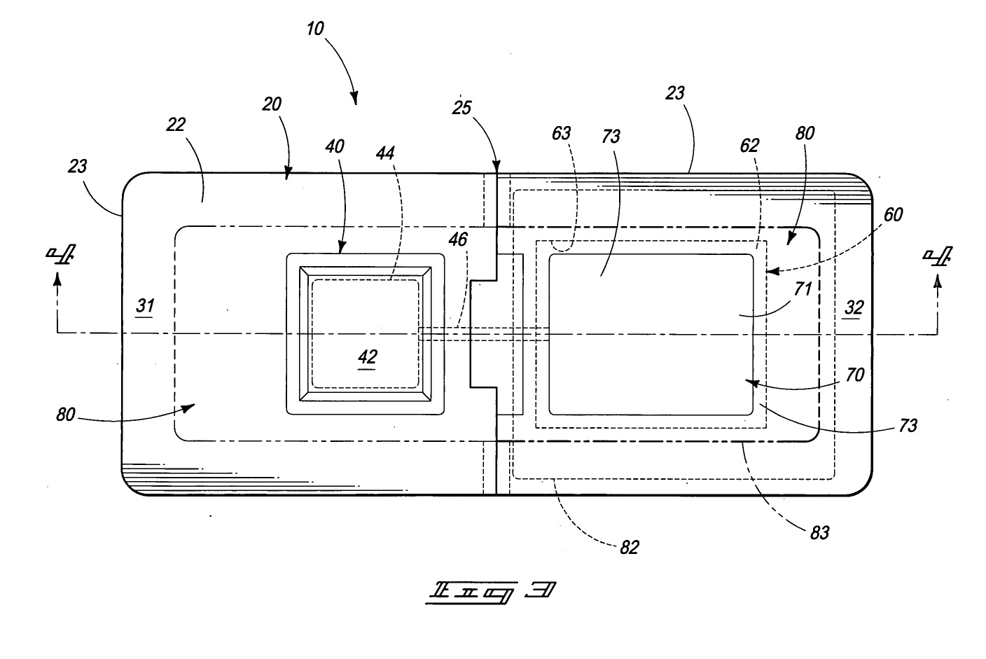

In addition to the foregoing, the present invention 10 includes an absorbent wound covering portion 70 for contacting a surface of a patient's body 11 and which is secured in covering relation relative to the patient's body 11 by the second portion 32 of the flexible substrate 20. In one form of the invention, the absorbent wound covering portion 70 carries a medicament 45 for treating a wound site 15 which is present on the surface of the patient's body. Still further, in another form of the invention, the absorbent wound covering portion 70 is affixed to, and carried by the inwardly facing surface 21 of the second portion 32 of the flexible substrate 20. In another form of the invention as seen in FIGS. 3 and 4, the at least partially rigid member is a pressure pad 40 which rests on a surface of a patient's body 11, and which further defines an internal cavity 44 which encloses a medicament or other treatment solution or emollient 45 for application to a wound site 15 which is formed on the surface of the patient's body 11. In this arrangement, the internal cavity 44 of the pressure pad 40 is coupled in fluid flowing relation relative to the second portion 32 of the flexible substrate 20. Still further, when a clinician 16 applies force to the pressure pad 40 it causes the medicament or other flowable substance 45 to flow from the internal cavity 44 of the pressure pad 40 to the surface of the patient's body 11 underlying the second portion 32 of the flexible substrate 20. Further, in this arrangement, the medicament or other treatment solution 45 may be partially absorbed by the absorbent wound covering portion 70, or otherwise may be delivered directly to the wound site 15. In the arrangement as seen in FIGS. 3 and 4, a conduit or passageway 50 is coupled in fluid flowing relation relative to the cavity 44 of the pressure pad 40, and provides a convenient means whereby a source of medicament or other flowable treatment fluid 51 is supplied by way of the conduit 50 to refill the cavity 44 of the pressure pad 40 with additional medicament 45.

The present invention also relates to a method for treating a wound site 15 which includes a first step of providing a flexible substrate 20 having opposite inwardly and outwardly facing surfaces 21 and 22, respectively, and which defines a first portion 31, and a second portion 32 which are moveably coupled together. The method includes another step of applying an adhesive 24 to at least a portion of the inside facing surface 21 of the flexible substrate 20. Still further, the method includes another step of mounting a pressure pad 40, on the inside facing surface 21 of the first portion 31 of the flexible substrate 20. The method further includes another step of positioning the pressure pad 40 in an anatomically correct position on a patient's body 11, and adhesively affixing the first portion of the flexible substrate 20 to the patients body 11 as seen in FIG. 5. Still further, after the step of positioning the pressure pad 40, the method includes another step of orienting the second portion of flexible substrate 20 in spaced relation relative to the patient's body 11 as seen in FIG. 5. After the step of orienting the second portion 32 of the flexible substrate 20 in spaced relation relative to the patient's body 11, the method includes another step of implementing a medical procedure which creates a wound site 15 in the patient's body 11 which is located in spaced relation relative to the pressure pad 40. Still further, the method includes another step of moving the second portion 32 of the flexible substrate 20 into covering relation relative to the wound site 15, and adhesively affixing it to the patient's body 11. Still further, the method includes a final step of positioning an absorbent wound covering portion 70 over the wound site 15, and in sandwiched relation therebetween the patient's body 11, and the inside facing surface 25 of the second portion 32 of the flexible substrate 20 prior to adhesively affixing the second portion 32 of the flexible substrate 20 to the patient's body 11.

In the present methodology, the method includes another step of providing individual releasable covers 80 which are located in covering relation relative to the inside facing surfaces 21 of the first and second portions 31 and 32 of the flexible substrate 20; and removing the respective releasable covers 80 prior to adhesively affixing the respective inside facing surfaces 21, of the first and second portions 31 and 32, to the patient's body 11. In the methodology of the present invention, the step of positioning an absorbent wound covering 70 over the wound site 15 further comprises attaching the absorbent wound covering 70 onto the inside facing surface 21 of the second portion 32 prior to the step of moving of the second portion 32 of the flexible substrate 20 into covering relation relative to the wound site 15. Additionally, in the present methodology the step of positioning an absorbent wound covering 70 over the wound site 15 further comprises a step of applying a medicament 45 to the absorbent wound covering 70 prior to covering the wound site 15 with the absorbent wound covering 70. In one form of the invention, the methodology comprises a step of applying a medicament 45 to the absorbent wound covering 70 after covering the wound site 15 with the absorbent wound covering 70. As seen in FIG. 4, the method of the present invention includes a further step of coupling the pressure pad 40 in fluid flowing relation relative to the inside facing surface 21 of the second portion 32 of the flexible substrate 20; and providing a source of a medicament 45 to be dispensed to the pressure pad 40. Still further, as seen in FIGS. 3 and 4, the method includes a step of coupling the pressure pad 40 to a source of the medicament 51 to be dispensed.

Therefore, it will be seen that the present invention provides a convenient means for treating a wound in a manner not possible, heretofore. A wound covering 10 is described herein which permits a medical procedure to be instituted on a patient in a convenient manner and thereafter a wound treated and then monitored through an optically transparent window in a manner not possible using the earlier prior art teachings.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

We claim:

1. A wound covering comprising:
a flexible substrate having first and second portions which are affixed together, and moveable one relative to the other, and wherein the second portion is at least in part optically transparent, and permits the viewing of a wound site on a patient; and
an at least partially rigid member mounted on the first portion, and extending outwardly relative to the first portion.

2. A wound covering as claimed in claim 1, and wherein the flexible substrate has an outwardly facing surface, and an inwardly facing surface which rests on a body surface of a patient, and wherein the at least partially rigid member is mounted on the inwardly facing surface of the first portion.

3. A wound covering as claimed in claim 2, and wherein the at least partially rigid member is moveable along an arcuately shaped path of travel relative to the flexible substrate.

4. A wound covering as claimed in claim 1, and wherein the flexible substrate has an outwardly facing surface, and an inwardly facing surface which rests on a body surface of a patient, and wherein the inwardly facing surface is covered at least in part by an adhesive which is effective in securing the flexible substrate to the body surface of the patient.

5. A wound covering as claimed in claim 1, and wherein the flexible substrate has an outwardly facing surface, and an inwardly facing surface which rests on a body surface of a patient, and wherein at least one of the inwardly and outwardly facing surfaces has a removable cover mounted thereon which maintains the inwardly and/or outwardly surface in a substantially sterile condition, and wherein the removable cover is removed prior to placing the flexible substrate in resting relation on the body surface of the patient.

6. A wound covering as claimed in claim 5, and wherein the removable cover includes a plurality of portions which can be separately removed while the remaining portions of the removable cover remain in covering relation relative to the inwardly and/or outwardly facing surface of the flexible substrate.

7. A wound covering as claimed in claim 5, and wherein the removable cover includes a plurality of portions which can be individually removed from, and then returned to, a covering relationship relative to the inwardly and/or outwardly facing surfaces of the flexible substrate while the remaining portions of the removable cover remain in covering relation relative to the inwardly and/or outwardly facing surface of the flexible substrate.

8. A wound covering as claimed in claim 1, and wherein the at least partially rigid member is a pressure pad which rests on a body surface of a patient, and wherein a clinician applies force to the pressure pad to achieve a desired medical outcome.

9. A wound covering as claimed in claim 1, and further comprising:
an absorbent wound covering portion for contacting a surface of a patient's body and which is secured in covering relation relative to the patient's body by the second portion of the flexible substrate.

10. A wound covering as claimed in claim 9, and wherein the absorbent wound covering portion carries a medicament for treating a wound which is present on the surface of the patient's body.

11. A wound covering as claimed in claim 9, and wherein the absorbent wound covering portion is affixed to, and carried by, the inwardly facing surface of the second portion of the flexible substrate.

12. A wound covering as claimed in claim 1, and wherein the at least partially rigid member is a pressure pad which rests on a surface of a patient's body, and which further defines an internal cavity which encloses a medicament for application to a wound which is on the surface of the patient's body, and wherein the internal cavity of the pressure pad is coupled in fluid flowing relation relative to the second portion of the flexible substrate, and wherein a clinician applies force to the pressure pad so as cause the medicament to flow from the internal cavity of the pressure pad to the surface of the patient's body underlying the second portion of the flexible substrate.

13. A wound covering as claimed in claim 12, and further comprising:
a conduit coupled in fluid flowing relation relative to the cavity of the pressure pad, and wherein a source of medicament is supplied to the conduit for refilling the cavity of the pressure pad with additional medicament.

14. A wound covering, comprising:
a flexible substrate having an inwardly facing surface which is coated at least in part by an adhesive, and an outwardly facing surface, and wherein the flexible substrate has a first and second portion, and wherein the second portion is, at least in part, optically transparent, and is operable to cover a site of a wound on a patient's body;
a pressure pad mounted on the inwardly facing surface of the first portion of the flexible substrate, and which is oriented in rested relation on a patient's body, and wherein a clinician applies force to the pressure pad to achieve a desired medical outcome; and
an absorbent wound covering portion which is oriented in covering relation relative to the site of the wound on the patient's body, and which is secured in place by the second portion of the flexible substrate.

15. A wound covering as claimed in claim 14, and wherein the second portion of the flexible substrate is spaced from the patient's body prior to the application of force to the pressure pad by the clinician, and wherein the clinician creates the wound in the patient's body, and applies the force to the pressure pad prior to creating the wound in the patient's body.

16. A wound covering as claimed in claim 14, and wherein the absorbent wound covering portion is mounted on, and carried by, the inwardly facing surface of the second portion of the flexible substrate.

17. A wound covering as claimed in claim 14, and wherein the absorbent wound covering is placed over the site of the wound on the patient's body by the clinician, and is sandwiched between the patient's body, and the inwardly facing surface of the second portion of the flexible substrate.

18. A wound covering as claimed in claim 14, and wherein individual, releasable covers are positioned in covering relation over the respective inside facing surfaces of the first and second portions, and are operable to maintain the inside facing surface substantially sanitary prior to being removed, and the inside facing surfaces of the respective first and second portions of the flexible substrate being place into contact with the patient's body.

19. A wound covering as claimed in claim 14, and wherein the pressure pad encloses a medicament which is supplied to the wound site when the clinician applies force to the pressure pad.

20. A wound covering as claimed in claim 14, and wherein the pressure pad moves independently of the first portion of the flexible substrate.

21. A wound covering as claimed in claim 14, and wherein the pressure pad defines an internal cavity which is coupled in fluid flowing relation relative to the inside facing surface of the second portion of the flexible substrate, and wherein the internal cavity encloses a medicament to be dispensed to the patient's body.

22. A wound covering as claimed in claim 19, and wherein the pressure pad is coupled in fluid receiving relation relative to a source of a medicament which is to be dispensed to the patient's body which is located under the second portion of the flexible substrate.

23. A method for treating a wound, comprising:
providing a flexible substrate having opposite inwardly and outwardly facing surfaces and which defines a first portion, and a second portion which are moveably coupled together;
applying an adhesive to at least a portion of the inside facing surface of the flexible substrate;
mounting a pressure pad on the inside facing surface of the first portion of the flexible substrate;
positioning the pressure pad in an anatomically correct position on a patient's body and adhesively affixing the first portion of the flexible substrate to the patients body;
after the step of positioning the pressure pad, orienting the second portion of flexible substrate in spaced relation relative to the patient's body;
after the step of orienting the second portion of the flexible substrate in spaced relation relative to the patient's body, implementing a medical procedure which creates a wound site in the patient's body which is located in spaced relation relative to the pressure pad;
moving the second portion of the flexible substrate into covering relation relative to the wound site and adhesively affixing it to the patient's body; and
positioning an absorbent wound covering portion over the wound site, and in sandwiched relation therebetween the patient's body, and the inside facing surface of the second portion of the flexible substrate prior to adhesively affixing the second portion of the flexible substrate to the patient's body.

24. A method as claimed in claim 23, and further comprising:
providing individual releasable covers which are located in covering relation relative to the inside facing surfaces of the first and second portions of the flexible substrate; and
removing the releasable covers prior to adhesively affixing the respective inside facing surfaces of the first and second portions to the patient's body.

25. A method as claimed in claim 23, and wherein the step of positioning an absorbent wound covering portion over the wound site further comprises attaching the absorbent wound covering portion onto the inside facing surface of the second portion prior to the step of moving of the second portion of the flexible substrate into covering relation relative to the wound site.

26. A method as claimed in claim 23, and wherein the step of positioning an absorbent wound covering portion over the wound site further comprises a step of applying a medicament to the absorbent wound covering portion prior to covering the wound site with the absorbent wound covering.

27. A method as claimed in claim 23, and wherein the step of positioning an absorbent wound covering over the wound site further comprises a step of applying a medicament to the absorbent wound covering portion after covering the wound site with the absorbent wound covering portion.

28. A method as claimed in claim 23, and further comprising:
coupling the pressure pad in fluid flowing relation relative to the inside facing surface of the second portion of the flexible substrate; and
providing a source of a medicament to be dispensed to the pressure pad.

29. A method as claimed in claim 28, and further comprising:
coupling the pressure pad to a source of the medicament to be dispensed.

30. A method as claimed in claim 23, and wherein the step of mounting a pressure pad on the inside facing surface of the first portion further comprises:
rendering the pressure pad independently moveable relative to the first portion of the flexible substrate.
